(19) United States
(12) Patent Application Publication
(10) Pub. No.: US 2022/0400667 A1
(43) Pub. Date: Dec. 22, 2022

(54) CRYOPRESERVATION DEVICES

(71) Applicant: CORNING INCORPORATED, Corning, NY (US)

(72) Inventors: John Wilfred Coddaire, Kennebunk, ME (US); William Joseph Lacey, North Andover, MA (US); Gregory Roger Martin, Acton, ME (US); Michael Kurt Schaefer, Gorham, ME (US); Paul Michael Szlosek, Kennebunk, ME (US); Allison Jean Tanner, Portsmouth, NH (US)

(21) Appl. No.: 17/896,697

(22) Filed: Aug. 26, 2022

Related U.S. Application Data
(62) Division of application No. 15/774,873, filed on May 9, 2018.
(60) Provisional application No. 62/255,627, filed on Nov. 16, 2015.

(51) Int. Cl. A01N 1/02; B65D 83/00; C12M 1/00; B01L 3/00

(52) U.S. Cl. CPC A01N 1/0268; B65D 83/0005; C12M 45/22; B01L 3/5082; B01L 3/5453; B01L 3/50825; B01L 2200/0689; B01L 2300/042; B01L 2300/0851; B01L 2300/123; B01L 2400/0481; B01L 2300/021; B01L 2200/141; B01L 2300/0832

(57) ABSTRACT

Vial assemblies comprising a tubular body and a flexible bottom or flexible liner are described herein, wherein the flexible bottom or liner is configured to compress at least partially upon the application of force. Methods for storing and removing frozen samples from such vial assemblies are also described herein.

This application claims the benefit of priority under 35 U.S.C. § 119 of U.S. Provisional Application Ser. No. 62/255,627 filed on Nov. 16, 2015 the content of which is relied upon and incorporated herein by reference in its entirety.

### FIELD OF THE DISCLOSURE

The present disclosure relates generally to cryopreseravation devices, and more specifically to cryogenic vial assemblies for preparing and storing frozen samples, and methods for removing frozen samples from such vial assemblies.

### BACKGROUND

Biological samples such as cells and tissues are often cryopreserved to extend their viability and usefulness for a variety of applications. For example, the cryopreservation process can involve placing a biological sample into an aqueous solution containing electrolytes and/or cryoprotectants and lowering the temperature of the solution to below its freezing point. Biological samples are often stored in vials which can be sealed and frozen, e.g., by immersion in liquid nitrogen. It can be important to maintain the sample integrity during the filling, storage, and retrieval stages, as contamination can render a biological sample useless for scientific research or other applications.

Vial leakage, which can be caused by a failure of the seal between the vial and the cap, can be a contributing factor to sample contamination. Sample contamination can also occur during thawing of the sample prior to its removal from the vial. For instance, cryogenic vials are often placed in a warm bath or heated block to partially or completely defrost the sample for ease of removal. However, samples can become contaminated or lose part of their viability during this process due to liquid immersion and/or elevated temperatures. The sample may also become overstressed due to excessive heating at the vial wall surface which can further damage the sample.

Removal of samples in the frozen state without thawing may reduce the risk of sample contamination and/or damage. However, it can be difficult to remove the frozen pellet from the vial due to adhesion of the sample to the vial walls and/or inability to grip and/or exert a force on the sample. Accordingly, it would be advantageous to provide vial assemblies from which frozen samples can be more easily discharged while also maintaining an acceptable sealed integrity for preventing sample contamination. It would also be advantageous to provide methods for preparing frozen samples which can be more easily discharged from a vial and methods for removing frozen samples from vials without the need for thawing prior to removal.

### SUMMARY

The disclosure relates, in various embodiments, to vial assemblies comprising a tubular body comprising a cavity, a first end, a second end for dispensing a sample, and a tube wall comprising a first material; and a flexible bottom comprising a second elastomeric material overmolded on an interior surface of the tube wall proximate the first end. Also disclosed herein are vial assemblies comprising a tubular body comprising a cavity, a first end, a second end, and a tube wall comprising a first material; and a flexible bottom comprising a second elastomeric material overmolded on the first end of the tubular body, wherein the flexible bottom further comprises at least two flanges attached to the tube wall and configured to hinge inwardly and apply force to a surface of the flexible bottom.

Further disclosed herein are vial assemblies comprising a substantially rigid tubular body comprising a cavity, a first end, a second end, and a tube wall comprising a first material; and a flexible liner comprising a second elastomeric material positioned in the cavity of the tubular body, wherein the flexible liner further comprises at least two flanges, and wherein the flanges are configured to hinge inwardly and apply force to a surface of the flexible liner. Still further disclosed herein are vial assemblies comprising a tubular body comprising a cavity, a first end, a second end for dispensing a sample, a tube wall, and a flexible bottom disposed on an interior surface of the tube wall proximate the first end, wherein the tube wall and flexible bottom comprise a first material, wherein the flexible bottom has a first thickness less which is less than a second thickness of the tube wall, and wherein the flexible bottom does not extend past the first end of the tubular body.

Methods for preparing and/or storing frozen samples in a vial assembly are also disclosed herein, as well as methods for removing such samples from the vial assemblies. Such methods can comprise removing a frozen sample from a vial assembly comprising a tubular body having a first end and an overmolded flexible bottom proximate the first end by applying force to at least a portion of the overmolded flexible bottom, wherein the flexible bottom is configured to compress at least partially upon the application of force. Removing a frozen sample from a vial assembly comprising a tubular body and a flexible liner inserted into a cavity of the tubular body can also comprise depressing two or more flanges of the flexible liner to apply force to at least a portion of the flexible liner, wherein the flexible liner is configured to compress at least partially upon the application of force. Finally, removing a frozen sample from a vial assembly comprising a tubular body comprising a tube wall, a first end, and a flexible bottom proximate the first end can comprise applying force to at least a portion of the flexible bottom, wherein the flexible bottom is configured to compress at least partially upon the application of force, and wherein the flexible bottom has a first thickness which is less than a second thickness of the tube wall.

Vial assemblies disclosed herein may provide a user with the ability to loosen and/or remove a frozen sample from a vial without thawing the sample beforehand. Such vial assemblies may also provide enhanced grip for the user when handling the frozen sample. Methods for removing frozen samples from the vial assemblies disclosed herein may have improved consistency and/or repeatability, thereby saving the user time and/or decreasing variability from sample to sample due to varying sample removal conditions, e.g., differing time and/or temperature used to thaw a sample. It should be noted, however, that one or more of such benefits may not be present according to various embodiments of the disclosure, yet such embodiments are intended to fall within the scope of the disclosure.

The disclosure provides, in an aspect (1) a vial assembly comprising a tubular body comprising a cavity, a first end, a second end for dispensing a sample, and a tube wall comprising a first material; and a flexible bottom comprising a second elastomeric material overmolded on an interior surface of the tube wall proximate the first end, wherein the flexible bottom does not extend past the first end of the tubular body. The disclosure further provides in an aspect (2), the vial assembly of aspect 1, wherein an interior surface of the tube wall proximate the flexible bottom comprises one or more features configured to retain an integral ejector. In an aspect (3), the disclosure provides the vial assembly of aspect 1 or 2, wherein the tube wall comprises at least two flanges proximate the flexible bottom, wherein the flanges are configured to hinge inwardly and apply force to the flexible bottom. In an aspect (4), the disclosure provides the vial assembly of any one of aspects 1-3, wherein the flanges further comprise at least one gusset configured to apply force to the flexible bottom.

In an aspect (5), the disclosure provides a vial assembly comprising: a tubular body comprising a cavity, a first end, a second end, and a tube wall comprising a first material; and a flexible bottom comprising a second elastomeric material overmolded on the first end of the tubular body, wherein the flexible bottom further comprises at least two flanges attached to the tube wall and configured to hinge inwardly and apply force to a surface of the flexible bottom.

In an aspect (6), the disclosure provides a vial assembly comprising: a substantially rigid tubular body comprising a cavity, a first end, a second end, and a tube wall comprising a first material; and a flexible liner comprising a second elastomeric material positioned in the cavity of the tubular body and extending past the first end of the substantially rigid tubular body, wherein the flexible liner further comprises at least two flanges, and wherein the flanges are configured to hinge inwardly and apply force to a surface of the flexible liner. In an aspect (7), the disclosure provides the vial assembly of aspect 6, wherein the second end comprises a lip and wherein the flexible liner covers at least a portion of the lip. In an aspect (8), the disclosure provides the vial assembly of aspect 6 or 7 further comprising a cap, wherein the flexible liner is configured to form a seal between the lip and the cap in a closed position. In an aspect (9), the disclosure provides the vial assembly of any one of aspects 6-8 wherein the flexible liner is overmolded on an interior or exterior surface of the tube wall.

In another aspect (10), the disclosure provides a vial assembly comprising: a tubular body comprising a cavity, a first end, a second end for dispensing a sample, a tube wall, and a flexible bottom disposed on an interior surface of the tube wall proximate the first end, wherein the tube wall and flexible bottom comprise a first material, wherein the flexible bottom has a first thickness less which is less than a second thickness of the tube wall, and wherein the flexible bottom does not extend past the first end of the tubular body. In an aspect (11), the disclosure provides the vial assembly of aspect 10, wherein at least a portion of the second end or at least a portion of the tube wall is overmolded with a third elastomeric material. In an aspect (12), the disclosure provides the vial assembly of aspect 11, wherein the third elastomeric material is patterned on an exterior surface of the tube wall and comprises at least one textured surface. In an aspect (13), the disclosure provides the vial assembly of aspect 11, wherein the third elastomeric material is overmolded around a periphery of the second end and on an exterior surface of the tube wall. In an aspect (14), the disclosure provides the vial assembly of aspect 13, wherein the exterior surface of the tube wall comprises at least one strip of the third elastomeric material extending from the second open along the entire length or a portion of the length of the tube wall. In an aspect (15), the disclosure provides the vial assembly of aspect 11, wherein the third elastomeric material is identical to or different from the second elastomeric material. In an aspect (16), the disclosure provides the vial assembly of aspect 10 or 11, wherein the tube wall comprises at least two flanges proximate the flexible bottom, wherein the flanges are configured to hinge inwardly and apply force to the flexible bottom.

In an aspect (17), the disclosure provides a method for removing a frozen sample from a vial assembly comprising a tubular body having a first end, a second end for dispensing the frozen sample, and a flexible bottom overmolded on an interior surface of the tubular body proximate the first end, the method comprising: applying force to at least a portion of the overmolded flexible bottom, wherein the flexible bottom is configured to compress at least partially upon the application of force. In a further aspect (18), the disclosure provides the method of aspect 17, wherein applying force to at least a portion of the overmolded flexible bottom comprises depressing two or more flanges proximate the first end. In an aspect (18), the disclosure provides the method of aspect 17 wherein applying force to at least a portion of the overmolded flexible bottom comprises manually depressing the overmolded flexible bottom. In an aspect (20), the disclosure provides the method of aspect 17 wherein applying force to at least a portion of the overmolded flexible bottom comprises bringing an integral ejector into contact with the overmolded flexible bottom. In an aspect (21), the disclosure provides a method for removing a frozen sample from a vial assembly comprising a tubular body and a flexible liner inserted into a cavity of the tubular body, the method comprising: depressing two or more flanges of the flexible liner to apply force to at least a portion of the flexible liner, wherein the flexible liner is configured to compress at least partially upon the application of force. In an aspect (22), the disclosure provides a method for removing a frozen sample from a vial assembly comprising a tubular body comprising a tube wall, a first end, and a flexible bottom disposed proximate the first end, the method comprising: applying force to at least a portion of the flexible bottom, wherein the flexible bottom is configured to compress at least partially upon the application of force, wherein the tube wall and flexible bottom comprise a first material, and wherein the flexible bottom has a first thickness which is less than a second thickness of the tube wall. In an aspect (23) the disclosure provides the method of aspect 22, wherein applying force to at least a portion of the flexible bottom comprises depressing two or more flanges proximate the first end. In an aspect (24), the disclosure provides the method of aspect 22, wherein applying force to at least a portion of the flexible bottom comprises manually depressing the flexible bottom.

Additional features and advantages of the invention will be set forth in the detailed description which follows, and in part will be readily apparent to those skilled in the art from that description or recognized by practicing the invention as described herein, including the detailed description which follows, the claims, and the appended drawings.

It is to be understood that both the foregoing general description and the following detailed description present various embodiments of the disclosure, and are intended to provide an overview or framework for understanding the nature and character of the claims. The accompanying drawings are included to provide a further understanding of the disclosure, and are incorporated into and constitute a part of this specification. The drawings illustrate various embodiments of the disclosure and together with the description serve to explain the principles and operations of the invention.

### BRIEF DESCRIPTION OF THE DRAWINGS

The following detailed description can be best understood when read in conjunction with the following drawings, in which, where possible, like numbers are used to refer to like elements, and:

FIGS. 1A-B are cross-sectional views of a vial assembly according to embodiments of the disclosure;

FIGS. 2A-C are cross-sectional views of a vial assembly according to various disclosed embodiments;

FIGS. 3A-B are cross-sectional views of a vial assembly according to certain embodiments of the disclosure;

FIGS. 4A-B are cross-sectional views of a vial assembly according to additional disclosed embodiments;

FIG. 5A is a cross-sectional view of the bottom of a vial assembly according to certain embodiments disclosed herein;

FIG. 5B is a perspective view of a vial assembly according to embodiments disclosed herein;

FIG. 6A is a perspective view of a vial assembly according to further embodiments of the disclosure;

FIG. 6B is a cross-sectional view of a vial assembly according to various disclosed embodiments;

FIG. 7A is a perspective view of a vial assembly according to additional disclosed embodiments; and

FIG. 7B is a cross-sectional view of a vial assembly according to certain disclosed embodiments.

### DETAILED DESCRIPTION

Vial Assemblies

Disclosed herein are vial assemblies comprising a tubular body comprising a cavity, a first end, a second, end for dispensing a sample, and a tube wall comprising a first material; and a flexible bottom comprising a second elastomeric material overmolded on an interior surface of the tube wall proximate the first end, wherein the flexible bottom does not extend past the first end of the tubular body. Also disclosed herein are vial assemblies comprising a tubular body comprising a cavity, a first end, a second end, and a tube wall comprising a first material; and a flexible bottom comprising a second elastomeric material overmolded on the first end of the tubular body, wherein the flexible bottom further comprises at least two flanges attached to the tube wall and configured to hinge inwardly and apply force to a surface of the flexible bottom.

Further disclosed herein are vial assemblies comprising a substantially rigid tubular body comprising a cavity, a first end, a second end, and a tube wall comprising a first material; and a flexible liner comprising a second elastomeric material positioned in the cavity of the tubular body and extending past the first end of the substantially rigid tubular body, wherein the flexible liner further comprises at least two flanges, and wherein the flanges are configured to hinge inwardly and apply force to a surface of the flexible liner. Still further disclosed herein are vial assemblies comprising a tubular body comprising a cavity, a first end, a second end for dispensing a sample, a tube wall, and a flexible bottom disposed on an interior surface of the tube wall proximate the first end, wherein the tube wall and flexible bottom comprise a first material, wherein the flexible bottom has a first thickness less which is less than a second thickness of the tube wall, and wherein the flexible bottom does not extend past the first end of the tubular body.

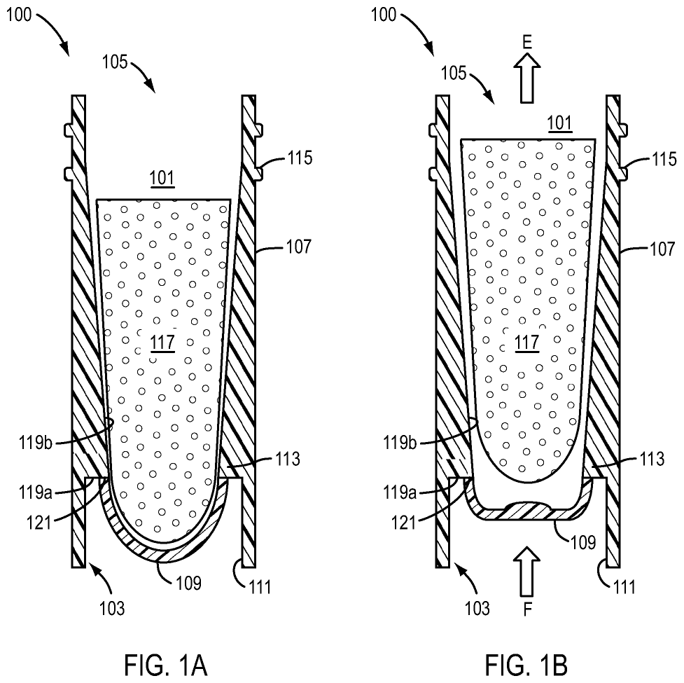

Embodiments of the disclosure will be discussed with reference to FIGS. 1-7, which illustrate various non-limiting aspects of a vial assembly according to the disclosure. Of course, it is to be understood that the vial assemblies are not drawn to scale and the relative size and/or proportion of the entire assembly and/or of portions of the assembly relative to other portions of the assembly can be smaller or larger than those depicted. The following general description is intended to provide an overview of the claimed devices and various aspects will be more specifically discussed throughout the disclosure with reference to the non-limiting embodiments, these embodiments being interchangeable with one another within the context of the disclosure.

As demonstrated in FIGS. 1A-B, which are cross-sectional views of a vial assembly according to various embodiments of the disclosure, a vial assembly can comprise a tubular body 100 including a cavity 101, a first end 103, a second end 105, and a tube wall 107. A flexible bottom 109 can be overmolded on an interior surface 111 of the tube wall 107 proximate the first end 103. In some instances, and as illustrated, the flexible bottom 109 can be overmolded onto an interior feature 113 on the tube wall 107 interior surface 111. In some embodiments, threading 115 may be present on an exterior or interior surface of the tube wall 107 proximate the second end 105, for securing a cap (not illustrated) thereto. As illustrated in FIG. 1A, the cavity 101 can contain a frozen sample or pellet 117. As illustrated in FIG. 1B, the sample 117 can be at least partially dislodged by application of force F to the flexible bottom 109, which can dislodge and/or eject the frozen sample in direction E, e.g., such that it can be dispensed via the second end 105.

The tubular body 100 can comprise, for example, a substantially cylindrical or frusto-conical cavity 101, which can be delineated by the tube wall 107 and the flexible bottom 109. The tube wall 107 may be constructed from any material suitable for a vial assembly such as a cryotube. For example, the tube wall may be constructed from a first material chosen from polyolefins (e.g., polyethylene or polypropylene), fluoropolymers (e.g., polytetrafluoroethylene), polycarbonates, polyesters, polystyrenes, and other similar polymers, or blends thereof. In some embodiments, the first material can comprise a substantially rigid or non-deformable material, such as a substantially rigid plastic material.

An interior surface 111 of the tube wall 107 proximate the first end 103 can be overmolded with a second material, which may be chosen from flexible elastomeric materials, to form the flexible bottom 109. Examples of suitable flexible or deformable elastomeric materials include, but are not limited to thermoplastic and thermoset elastomers, such as silicon-based polymers (e.g., polydimethylsiloxane) or multi-block elastomer alloys (e.g., Versaflex™ products from PolyOne, such as CL2250 or HC MT222), to name a few. According to various embodiments, the first material can have a higher rigidity than that of the second material, e.g., the tubular body 107 can be substantially rigid while the bottom 109 can be substantially flexible and/or deformable.

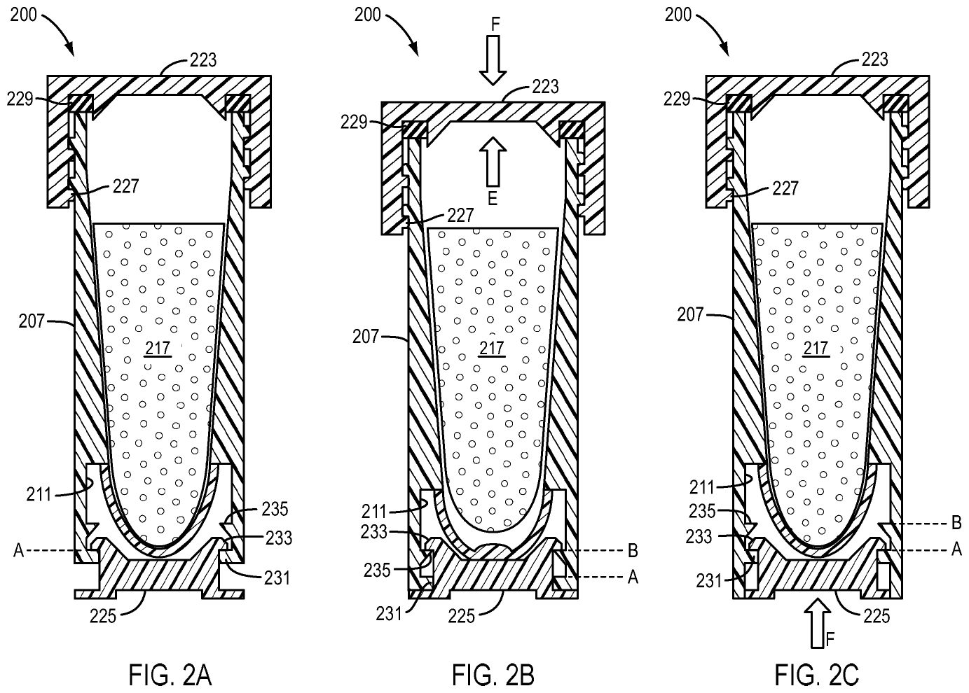

As used herein the term “end” is intended to refer to an edge or boundary of tubular body which may be open or closed. For instance, the second end can be open or closed by a cap. Similarly, the first end can be closed by a flexible bottom or other feature. The flexible bottom can be recessed inside the tubular body or can protrude past the first end. Referring to FIGS. 1A-B, the tubular body can be “closed” by the overmolded flexible bottom recessed inside the tubular body proximate the first end, while the first end can remain “open” such that the flexible bottom can be contacted by the user to eject the frozen sample. Referring to FIGS. 2A-C, the tubular body can be “closed” by the overmolded flexible bottom recessed inside the tubular body proximate the first end, and the first end can also be “closed” by the integral ejector feature.

The term “overmolded” is intended to denote that the second material is applied and bonded to the first material, e.g., by melt processing and subsequent bonding, or vice versa. The bond between the first and materials can be a mechanical, melt, and/or chemical bond, according to various embodiments. Mechanical bonding can entail, for instance, softening or melting the second material and flowing the material onto a substrate (e.g., a tubular body) comprising the first material, wherein the second material flows into holes, undercuts, channels, or other features on the substrate surface. After solidifying, the overmolded second material can thus be mechanically connected or interlocked with the substrate or tubular body. In certain instances, the overmolded material can form a mechanical lock, such as an interlocking tongue and groove, which can have a relatively high strength. Additionally, the use of mechanical bonding can provide a wider range of choices with respect to the first and second materials (e.g., in terms of melting temperatures, chemical composition, etc.).

Melt bonding can be derived from adhesion between the surfaces of two materials with relatively similar melting points. For example, at a given overmolding temperature, both materials may at least partially melt and mutually solvate at their interface to form a bond. Some degree of chemical similarity may enhance a melt bond thus formed, although it is not necessary. Chemical bonding can occur, for example, in the case of two chemically compatible materials, by a chemical reaction at the interface to bond the materials together. Chemical and melt bonding can provide a relatively strong bond between the two materials as compared to mechanical bonding but may also, in some instances, narrow the range of choices for the first and second materials.

Overmolding can be achieved, for example, by a two-shot molding process. In such a process, a first injection molding machine can be used to mold a substrate (e.g., tubular body) from the first material. The substrate can then be transferred to a second injection molding machine in which the second material is molded onto the substrate. In various embodiments, the substrate may have an elevated temperature above room temperature (from the first injection molding step), and may be in a semi-solid or gel phase. Thus, melt and chemical bonding may be improved between the first and second materials using such a two-shot molding process. Of course, the substrate can also be provided with features to provide one or more mechanical bonds or locks.

According to various embodiments, the flexible bottom 109 can be overmolded onto an interior surface 111 of the tube wall 107. In some embodiments, the second material can be molded directly on to the interior surface. In other embodiments, the second material can be molded onto a feature 113 of the interior surface (as shown in FIGS. 1A-B). As such, as used herein, the term “interior surface” of the tube wall is intended to denote an interior surface of the tube wall, a feature on the interior surface, a surface of the feature, or any combination thereof.

The feature 113 can, in various embodiments, be constructed of the same material as the tube wall. The feature 113 can be a protrusion or recess having any desired shape or size. For example, the feature 113 can have a wedge-shaped cross-section (as depicted) such that a portion of the feature proximate the first end 103 can extend or jut into the cavity 101. Of course any other feature shape and/or size is possible and envisioned as falling within the scope of the disclosure. Any surface of the feature 113 can be used as a bonding interface between the first material and the second material. For instance, as illustrated in FIGS. 1A-B, the feature can comprise a surface 119a forming an angle (e.g. a perpendicular angle) with to the tube wall 107 and this surface can comprise the bonding interface 121. It should be noted that surface 119b, also forming an angle with the tube wall 107, could also be used as a bonding interface, or any other portion of the interior surface 111 of the tube wall 107, without limitation.

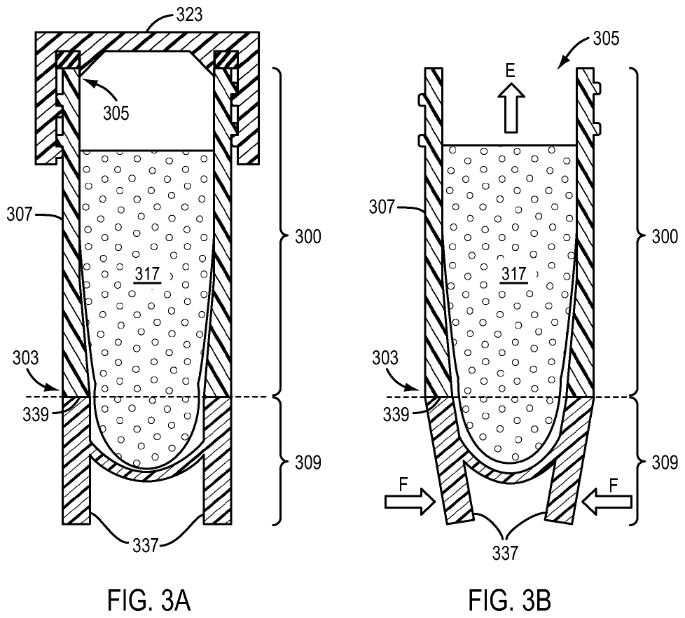

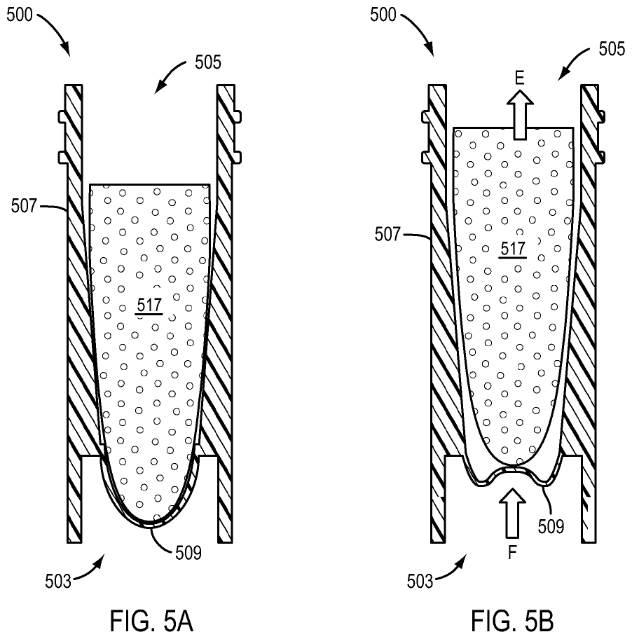

As depicted in FIG. 1B, a force F can be applied to the flexible bottom 109 to dislodge or eject a sample 117 from the vial assembly. In certain embodiments, a component external to the vial assembly can be brought into contact with the flexible bottom to apply the force F. For example, a user can depress the flexible bottom with a thumb or finger, or with a separate device, such as a plunger, ejector, or any other suitable object. In other embodiments, an ejector may be integral to the vial assembly, for instance, as depicted in FIGS. 2-4 and 6. The ejector can be a separate piece fitted to the vial assembly (e.g., FIGS. 2A-C) or can be a part of the tubular body, e.g., the tube wall (e.g., FIGS. 5A-B). In other embodiments, the ejector can be part of or otherwise attached to the flexible bottom (e.g., FIGS. 3A-B and 4A-B).

FIGS. 2A-C depict cross-sectional views of a vial assembly of FIGS. 1A-B further equipped with a cap 223 and an integral ejector 225. In some embodiments, the cap 223 can comprise threading 227 on an internal or external surface, configured to mate with threading 215 on the tube wall 207. A seal 229 may also be present to enhance and/or tighten the closure between the tubular body and the cap. The cap 223 and/or ejector 225 can be constructed from any suitable material, for example, materials similar to or different from the material from which the tube walls 207 are constructed. In non-limiting embodiments, the cap and/or ejector can comprise a first material chosen from polyolefins (e.g., polyethylene or polypropylene), fluoropolymers (e.g., polytetrafluoroethylene), polycarbonates, polyesters, polystyrenes, and other similar polymers, or blends thereof. Of course the additional depicted features in FIGS. 2A-C are not limited to operation with the vial of FIGS. 1A-B and any features disclosed herein can be interchanged with any other vial assembly.

Vial assemblies may be equipped with bar codes for tracking samples, for example, a bar code may be placed on the bottom of a cryotube. Bar codes can be printed, for example, on a bar code disk that can be located at the base of the tube. In some embodiments, a bar code disk can be used as an integral ejector 225. Of course, an integral ejector having a different shape and/or size can be used, with or without the presence of a barcode, these features being interchangeable without limitation. The integral ejector 225 can be retained in a first position A, in which the ejector does not apply an ejecting force on the flexible bottom. The interior surface 211 of the tube wall 207 can, for example, be equipped with one or more features 231 for retaining the ejector 225. Such features can comprise protrusions or recesses on or in the tube wall 207. As illustrated, protrusions jut out from the interior surface 211; however, a different configuration in which the inner surface is contoured to create recesses is also envisioned, as well as any other feature capable of retaining the ejector 225. The integral ejector 225 can comprise one or more corresponding features 233 configured to hold or retain the ejector in the desired position (e.g., position A as depicted in FIG. 2A).

As shown in FIG. 2B, a force F can be applied to the top of the vial assembly such that the integral ejector 225 is dislodged from the first position and forced into a second position B, in which the ejector contacts the flexible bottom thereby applying an ejecting force to the sample 217 in direction E, e.g., dispensing the sample via second end 205. For example, a user can press down on the vial cap 223, or up on the ejector 225 to generate an ejecting force. In some embodiments, one or more second features 235 can be included on the interior surface 211 to retain the ejector 225 in position B. As depicted in FIG. 2C, the integral ejector 225 can be configured to fully nest within the tube wall 207, in which case force F can be applied to the ejector, e.g., by depressing or pushing the ejector inward. Alternatively, as depicted in FIGS. 2A-B, the ejector 225 can be configured to extend at least partially beyond the tube wall 207, in either or both positions A or B, in which case force F can be applied to the top or bottom of the vial assembly to eject the sample.

Additional exemplary vial assemblies are depicted in FIGS. 3A-B. In the illustrated embodiments, the vial assembly comprises a tubular body 300 comprising a first end 303, a second end 305 (labeled in FIG. 3B, closed by cap 323 in FIG. 3A), and a tube wall 307. A flexible bottom 309 is overmolded on the first end 303 (boundary indicated by the dashed line), e.g., extending across the tube wall and closing the first end of the tubular body 300. The flexible bottom 309 can be equipped with two or more flanges 337, which can be configured to serve multiple functions, e.g., providing a stand for the vial and/or a means for storing the vial within a rack or stand. As shown in FIG. 3B, the flanges 337 can be configured to bend inwardly upon application of force F to apply pressure to the flexible bottom 309. For instance, the flanges 337 may be used as a hinge to pinch or otherwise impinge upon the flexible bottom 309 such that the frozen sample 317 is dislodge or ejected from the vial in direction E, e.g., dispensing the sample from the second end 305.

As discussed above, the tubular body can comprise a first material and the flexible bottom can comprise a second elastomeric material overmolded on the first opening of the tubular body. In some embodiments, a bottom outer surface 339 (see FIG. 3B) of the tube wall 307 can serve as the bonding interface (not labeled), although the flexible bottom can also be overmolded on other surfaces, such as the interior or exterior surfaces of the tube wall 307. The flexible bottom 309 can, for instance, span the entire periphery of the first end 303 of the tubular body. In some embodiments, as illustrated, the flexible bottom 309 and flanges 337 can form a unitary overmolded piece. The flanges 337 can extend, e.g., from the bottom surface 339 of the tube wall 307 and can, in some instances, form a full or partial extension of the tube wall 307. For example, each flange can be semi-circular or arcuate in shape, e.g., approximating the peripheral contour of the tube wall 307, and can provide a stand for the vial assembly such that the flexible bottom 309 is not impinged upon by a surface on which the vial rests when in the upright position.

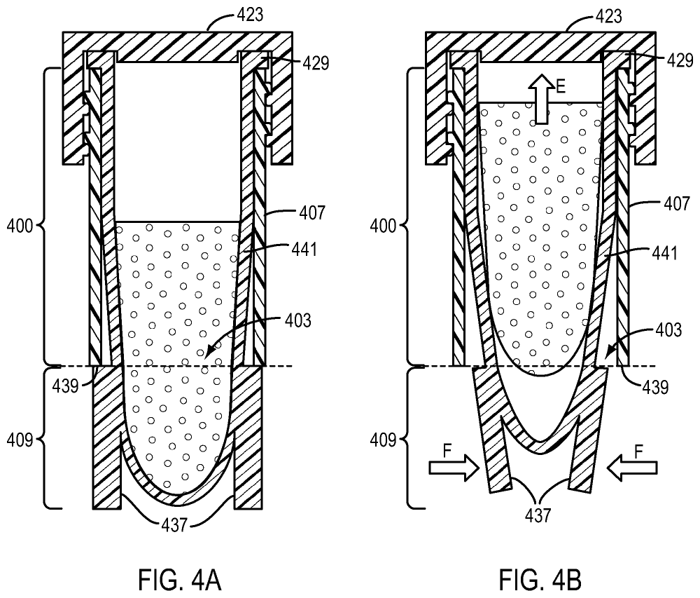

In further embodiments, as illustrated in FIGS. 4A-B, the vial assembly can comprise a tubular body 400 comprising a first end 403, a second end 305 (not labeled, closed by cap 423), and a tube wall 407. A flexible liner 441 can be inserted into or otherwise positioned in the cavity (not labeled) of the tubular body. In some non-limiting embodiments, the liner 441 can be overmolded onto an interior and/or exterior surface of the tube wall 407. The flexible liner 441 can extend past the first end 403 (boundary indicated by the dashed line) of the tubular body 400, e.g., providing a flexible bottom 409 closing the first end of the tubular body 400.

As in FIGS. 3A-B, the flexible bottom 409 can be equipped with two or more flanges 437, which can be configured to serve multiple functions, e.g., providing a stand for the vial and/or a means for storing the vial within a rack or stand. As shown in FIG. 4B, the flanges 437 can be configured to bend inwardly upon application of force F to apply pressure to the flexible bottom 409. For instance, the flanges 437 may be used as a hinge to pinch or otherwise impinge upon the flexible bottom 409 such that the frozen sample 417 is dislodge or ejected from the vial in direction E. Furthermore, as illustrated in FIG. 4B, the cap need not be removed to exert force on the flexible bottom to at least partially dislodge the frozen sample or pellet. As such, for any vial assemblies disclosed herein, the cap can be removed followed by applying force to the flexible bottom or vice versa.

The tubular body can comprise a first material and the flexible liner can comprise a second elastomeric material inserted into the cavity of the tubular body. In some embodiments, the flexible liner 441 can also serve as a seal 429 between a top surface (not labeled) of the tubular body and the cap 423. For instance, the second end (not labeled) can comprise a lip which can engage the flexible liner 441, e.g., such that the liner sits atop, rests on, or abuts the lip. In the closed position, e.g., when the cap 423 is engaged to the tubular body via threading or another closing feature, at least a portion of the flexible liner 441 can rest between the cap 423 and tubular body 400 to enhance the integrity of the vial assembly. Suitable elastomeric materials can include, for example, thermoplastic and thermoset elastomers, such as silicon-based polymers (e.g., polydimethylsiloxane) or multi-block elastomer alloys (e.g., Versaflex™ products from PolyOne, such as CL2250 or HC MT222), to name a few.

In some embodiments, as illustrated, the flexible liner 441, bottom 409, and flanges 437 can form a unitary piece. The flanges 437 can extend, e.g., from the bottom surface 439 of the tube wall 407 and can, in some instances, form a full or partial extension of the tube wall 407. For example, each flange can be semi-circular or arcuate in shape, e.g., approximating the peripheral contour of the tube wall 407, and can provide a stand for the vial assembly such that the flexible bottom 409 is not impinged upon by a surface on which the vial rests when in the upright position.

In alternative embodiments, as illustrated in FIGS. 5A-B, a vial assembly can comprise a unitary piece constructed from a single (e.g., first) material. Similar to the other vial assemblies, a tubular body 500 can comprise a first end 503, a second end 505, a tube wall 507, and a flexible bottom 509. As depicted in FIG. 5B, a force F can be applied to the flexible bottom 509 to dislodge or eject a sample 517 from the vial assembly. In certain embodiments, a component external to the vial assembly can be brought into contact with the flexible bottom to apply the force F. For example, a user can depress the flexible bottom with a thumb or finger, or with a separate device, such as a plunger, ejector, or any other suitable object. In other embodiments, an ejector may be integral to the vial assembly.

In non-limiting embodiments, the tubular body 500, e.g., flexible bottom 509 and tube wall 507, can comprise a first material chosen from polyolefins (e.g., polyethylene or polypropylene), fluoropolymers (e.g., polytetrafluoroethylene), polycarbonates, polyesters, polystyrenes, and other similar polymers, or blends thereof. In certain embodiments, the tubular body 500 can comprise a material which, at a sufficiently high thickness can provide rigidity (e.g., for the tube wall 507), but at a sufficiently low thickness can provide flexibility (e.g., for the bottom 509). For instance, various portions of the tubular body 500 can have a reduced wall thickness to enhance malleability of the tube in those locations, e.g., at the bottom. Whereas the tube wall 507 can have a thicker sidewall, the bottom 509 may have a reduced wall thickness, which may facilitate bending, flexing, and/or pinching of the tube in this location to encourage dislodgment of the frozen sample from the tube.

According to various embodiments, the thickness of the tube wall 507 can range from about 0.05 cm to about 0.2 cm, such as from about 0.06 cm to about 0.15 cm, or from about 0.075 cm to about 0.125 cm, including all ranges and subranges therebetween, whereas the thickness of the bottom 509 can range from about 0.025 cm to about 0.1 cm, such as from about 0.03 cm to about 0.075 cm, from about 0.04 cm to about 0.07 cm, or from about 0.05 cm to about 0.06 cm, including all ranges and subranges therebetween. In certain embodiments, a ratio of the thickness of the tube wall to the thickness of the bottom can range from about 2:1 to about 20:1, such as from about 3:1 to about 15:1, from about 4:1 to about 12:1, from about 5:1 to about 10:1, or from about 6:1 to about 8:1, including all ranges and subranges therebetween.

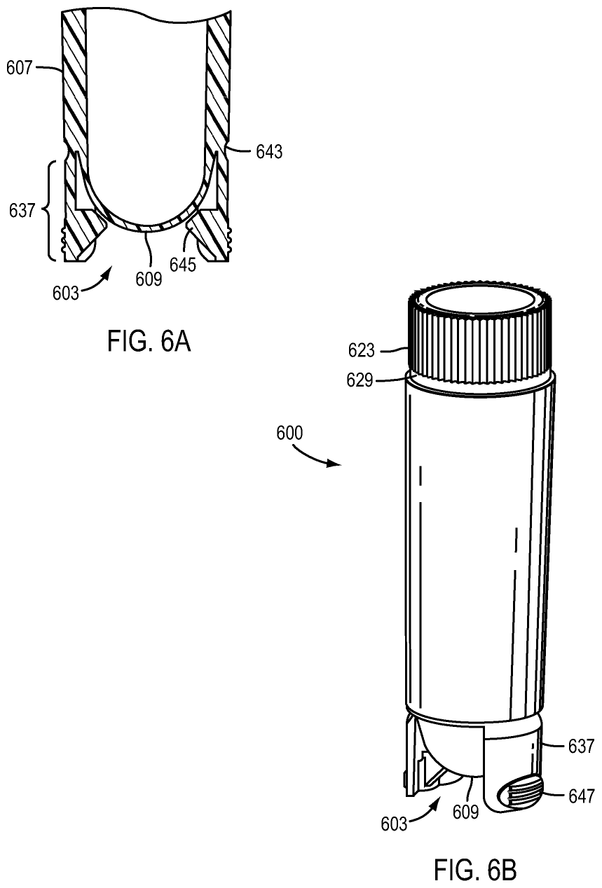

Whether the tubular body comprises one material (e.g., as illustrated in FIGS. 5A-B) or more than one material (e.g., as illustrated in FIGS. 1A-B), the first end of the tubular body can be equipped with one or more features for promoting disengagement of the frozen sample from the vial assembly. The first end of the tubular body 600 and will be discussed in more detail with reference to the cross-sectional image depicted in FIG. 6A. In some embodiments, the vial assembly can be provided with flanges 637 proximate the first end 603. As illustrated in FIG. 6A, the bottom 609 can be substantially rounded, although other shapes are possible and envisioned as falling within the scope of the disclosure. The flanges 637 can be part of the tubular body (as illustrated), e.g., constructed from the same material, or separate from the tubular body, e.g., constructed from a different material. According to various embodiments, the flanges 637 can be configured to bend inwardly upon application of force to apply pressure to the bottom 609. For instance, the flanges 637 may be used as a hinge to pinch or otherwise impinge on the bottom 609.

Further features may be added to the flanges 637 to enhance the ability to dislodge the frozen sample from the vial. In some embodiments, the tube wall 607 of the vial can be thinned in a region 643 proximate the bottom 609. Thinning of the tube wall 607 in this location may enhance the ability of the flanges 637 to hinge inwardly. Additionally, the flanges 637 can be equipped with gussets or inward protrusions 645 that can increase the force applied to the bottom 609. Lateral or perpendicular force applied to the flanges 637 can be redirected by the gussets 645 to the bottom, e.g., providing a vertical or parallel force pushing into the bottom 609 to dislodge the pellet.

FIG. 6B illustrates a perspective view of the exterior of a vial assembly according to various aspects of the disclosure. In the closed position, the cap 623 can be engaged with the tubular body 600 via seal 629. The first end 603 of the tubular body 600 is optionally equipped with flanges 637 for standing the vial upright and/or for disengaging the frozen sample from the vial. The flanges 637 can, in some embodiments, include a textured region 647, which can be raised for enhanced gripping by a user during use (e.g., finger grips) and/or for insertion into a storage rack or block, or recessed for fitting into standard tube racks.

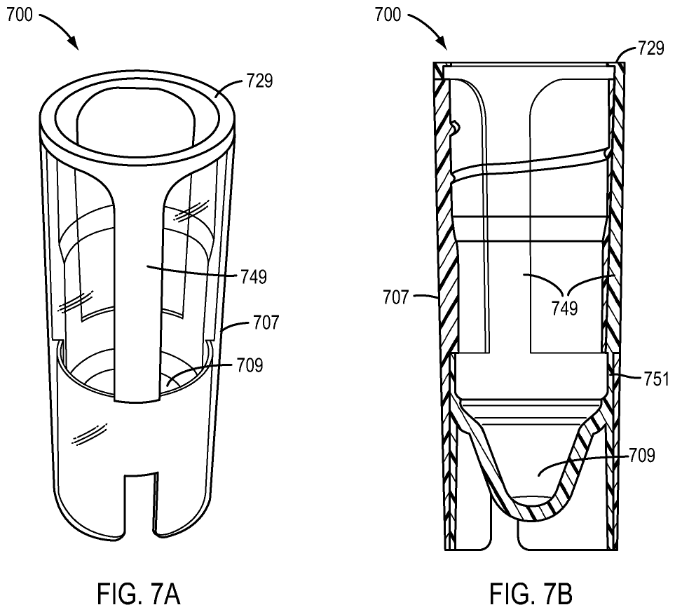

Gripping features can, in some embodiments, advantageously enable a user to hold onto the vial assembly during handling and/or opening. For instance, slippery condensate on the frozen vial and/or a loss of dexterity due to personal protection equipment worn by the user can make handling and/or opening the vial assembly difficult. According to various embodiments, it may be advantageous to provide gripping or textured features on the exterior of the tubular body as shown in FIGS. 7A-B.

Furthermore, as discussed above, a seal is often incorporated to improve closure of the vial between the tubular body and the cap. Currently, a secondary process is employed to add a seal or gasket to the vial assembly. However, such secondary seals tend to become brittle when exposed to cryogenic conditions, such as liquid nitrogen. Moreover, the additional processing steps can complicate the process and/or increase the expense of the final product. It would thus additionally be advantageous to provide sealing features during an overmolding step, e.g., during formation of a flexible bottom and/or gripping features on the vial assembly, using materials with enhanced ductility and/or reduced brittleness.

Such gripping and sealing features will be discussed with reference to FIGS. 7A-B, which depict perspective and cross-sectional views of a vial assembly according to various non-limiting embodiments. According to some embodiments, such as the vial assemblies depicted in FIGS. 1-3 including overmolded flexible bottoms, the overmolding process can also be used to supply gripping and/or sealing features on the exterior of the tubular body. For example, as illustrated in FIGS. 7A-B, during an overmolding process used to bond the flexible bottom 709 to the tube wall 707, the second material can also be overmolded on one or more portions of the tube wall exterior, such as gripping features 749 and/or sealing features 729.

The sealing and/or gripping features 729, 749 can be bonded to the tubular body 700 via mechanical, chemical, and/or melt bonding. In some embodiments, the gripping features 749 can be at least partially mechanically bonded to the tube wall 707 via one or more channels, grooves, or troughs (not illustrated) into which the second material can flow after being melted or softened. For instance, during an overmolding process, the flexible bottom can be formed at the first end and the second material can then flow along one or more troughs or channels created on a surface of the tubular body. According to various embodiments, after forming the flexible bottom at the first end, a band 751 can be formed along an internal or external periphery of the tube wall, e.g., by flowing the second material into a trough formed on a surface of the tube wall (interior surface illustrated). One or more gripping features 749, which may be optionally textured, can then be formed by flowing the second material into one or more grooves or channels running along the length or a portion of the length of the exterior surface of the tube wall. Finally, a sealing feature or gasket 729 can be formed by flowing the second material over the outer periphery or lip of the second end. In this non-limiting embodiment, the surface area of attachment and the various mechanical interlocking joints formed by the channels, troughs, and/or gasket can create a very strong bond between the first and second materials.

While FIGS. 7A-B illustrate three gripping features 749 substantially spaced apart and having substantially the same shape, it is to be understood that any shape and/or number of features can be provided using different channels, grooves, and/or troughs. In some embodiments, one or two gripping features can be included, or more than three gripping features can be included, such as four, five, six, or more features. Additionally, while gripping features 749 are illustrated as extending along the entire length of the tubular body, it is to be understood that such features can have any desired length. Moreover, while FIGS. 7A-B illustrate such features in combination with a vial assembly comprising an overmolded flexible bottom, these features can also be combined with other vial assemblies disclosed herein, such as the embodiments illustrated in FIGS. 4A-B or 5A-B.

The vial assemblies, including the tubular bodies, caps, liners, bottoms, grips, etc., can be manufactured from any materials suitable for cryopreservation applications, for instance, the first and second materials disclosed herein. In some embodiments, the first and/or second material can be a thermally conductive material, e.g., a material capable of transferring heat from a user's hands or another heat source to the frozen sample to further facilitate removal of the sample from the vial assembly. According to various embodiments, the tubular body and cap of the vial assembly can comprise the same or different materials. In additional embodiments, the tube wall and flexible bottom of the tubular body can comprise the same or different materials. The cap may comprise a substantially rigid material, whereas the tube wall can comprise a similarly rigid or relatively less rigid material. Likewise, the flexible bottom can comprise a material having less rigidity as compared to the tube wall and/or cap.

Additional optional features can be included in the vial assembly disclosed herein, e.g. for improved ease of handling, heat transfer, and/or sealing of the vial. Some exemplary non-limiting features are described, for example, the caps disclosed in U.S. Provisional Application No. 62/255,633 entitled “CRYOGENIC VIAL ASSEMBLIES” filed on Nov. 16, 2015, which is incorporated herein by reference in its entirety.

Methods

Methods disclosed herein can include methods for preparing a frozen sample in a vial assembly and methods for removing a frozen sample from a vial assembly. Methods for preparing and/or storing frozen samples can include introducing a liquid sample into a cavity of a tubular body; engaging an open end of the tubular body with a cap to form a sealed vial assembly; and freezing the sealed vial assembly. Methods for removing frozen samples from a vial assembly can include applying force to the flexible bottom of the vial assembly to loosen and/or dislodge the frozen sample from the tube. Force can be applied, for example, using an external ejector such as a thumb or finger of the user or any other component, such as a plunger or other similar tool. Force can also be applied using an integral ejector, e.g., part of the vial assembly, such as an ejector retained by one or more features of the vial assembly and/or flanges making up one or more parts of the assembly. Of course, it is to be understood that the features disclosed herein with respect to the vial assembly are intended to similarly apply to the methods disclosed herein, such that a method for preparing or removing the sample can employ or utilize one or more features described with respect to the vial assembly.

According to various embodiments, a frozen sample, e.g. comprising a biological sample such as cells or tissues, can be introduced into a vial assembly disclosed herein. For instance, a predetermined amount of a liquid sample can be poured the second end of the tubular body. The liquid sample can be added, in various embodiments, in any desired amount. By way of non-limiting example, the liquid sample can at least partially fill the cavity of the tubular body, e.g., at least about 10%, 20%, 30%, 40%, 50%, 60%, 70%, 80%, 90%, or even 100% of the cavity may be filled with the liquid sample.

A cap can then be coupled to the second end of the tubular body, e.g., by rotating the cap such that threading on the cap engages at least a portion of threading on the tubular body, by snap-fit, or by a like mechanism. Coupling of the cap to the tubular body, e.g., by rotation, can be carried out until the cap is snugly fit to the tubular body, for instance, until a bottom surface of the cap abuts a top surface or lip of the tubular body or until the cap abuts a seal disposed between the cap and the lip of the tubular body. Upon sealing the vial assembly in the closed position, the vial assembly and sample contained therein can be frozen, e.g., at a temperature below the freezing point of the sample. The vial assembly can, for instance, be frozen in an upright position such that the liquid is in contact with a bottom internal surface of the cavity. The vial assembly can also be frozen in any other position, such as a horizontal or inverted position, such that the liquid is in contact with a top internal surface and/or the bottom internal surface of the cavity. Regardless of orientation, the sample can be frozen such that application of force to the flexible bottom at least partially dislodges the sample from the vial assembly.

Removal of the frozen sample from the vial assembly can be achieved by disengaging the cap from the tubular body either before or after applying force to the flexible bottom to dislodge the frozen sample from the interior surfaces of the vial assembly. According to various embodiments, the flexible bottom can be compressed by squeezing two or more flanges proximate the closed end. The flanges can apply lateral and/or vertical (e.g., inward and/or upward) force to the bottom of the tubular body to push the frozen sample out of the vial. An integral or external ejector can also be pressed against the flexible bottom to push the frozen sample out of the vial, either by pushing the ejector into the flexible bottom or otherwise pressing the flexible into the ejector. The application of vertical and/or lateral force can serve to loosen the frozen sample from the internal surfaces of the tubular body such that the sample can be more easily ejected from the vial assembly.

It will be appreciated that the various disclosed embodiments may involve particular features, elements or steps that are described in connection with that particular embodiment. It will also be appreciated that a particular feature, element or step, although described in relation to one particular embodiment, may be interchanged or combined with alternate embodiments in various non-illustrated combinations or permutations.

It is also to be understood that, as used herein the terms “the,” “a,” or “an,” mean “at least one,” and should not be limited to “only one” unless explicitly indicated to the contrary. Thus, for example, reference to “an opening” includes examples having two or more such “openings” unless the context clearly indicates otherwise.

While labels such as “first” and “second” or “top” and “bottom” are used herein for ease of differentiating similar features, it is to be understood that these labels are not limiting on the disclosure or the appended claims and that such labels can be switched without limitation to refer to other similar features, unless the context clearly indicates otherwise.

Ranges can be expressed herein as from “about” one particular value, and/or to “about” another particular value. When such a range is expressed, examples include from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint.

All numerical values expressed herein are to be interpreted as including “about,” whether or not so stated, unless expressly indicated otherwise. It is further understood, however, that each numerical value recited is precisely contemplated as well, regardless of whether it is expressed as “about” that value. Thus, “a dimension less than 10 mm” and “a dimension less than about 10 mm” both include embodiments of “a dimension less than about 10 mm” as well as “a dimension less than 10 mm.”

Unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is no way intended that any particular order be inferred.

While various features, elements or steps of particular embodiments may be disclosed using the transitional phrase “comprising,” it is to be understood that alternative embodiments, including those that may be described using the transitional phrases “consisting” or “consisting essentially of,” are implied. Thus, for example, implied alternative embodiments to a method comprising A+B+C include embodiments where a method consists of A+B+C, and embodiments where a method consists essentially of A+B+C.

It will be apparent to those skilled in the art that various modifications and variations can be made to the present disclosure without departing from the spirit and scope of the disclosure. Since modifications combinations, sub-combinations and variations of the disclosed embodiments incorporating the spirit and substance of the disclosure may occur to persons skilled in the art, the disclosure should be construed to include everything within the scope of the appended claims and their equivalents.

1-5. (canceled)

6. A vial assembly comprising:
a substantially rigid tubular body comprising a cavity, a first end, a second end, and a tube wall comprising a first material; and
a flexible liner comprising a second elastomeric material positioned in the cavity of the tubular body and extending past the first end of the substantially rigid tubular body, wherein the flexible liner further comprises at least two flanges, and wherein the flanges are configured to hinge inwardly and apply force to a surface of the flexible liner.

7. The vial assembly of claim 6, wherein the second end comprises a lip and wherein the flexible liner covers at least a portion of the lip.

8. The vial assembly of claim 6, further comprising a cap, wherein the flexible liner is configured to form a seal between the lip and the cap in a closed position.

9. The vial assembly of claim 6, wherein the flexible liner is overmolded on an interior or exterior surface of the tube wall.

10. A vial assembly comprising:
a tubular body comprising a cavity, a first end, a second end for dispensing a sample, a tube wall, and
a flexible bottom disposed on an interior surface of the tube wall proximate the first end,
wherein the tube wall and flexible bottom comprise a first material,
wherein the flexible bottom has a first thickness less which is less than a second thickness of the tube wall, and
wherein the flexible bottom does not extend past the first end of the tubular body.

11. The vial assembly of claim 10, wherein at least a portion of the second end or at least a portion of the tube wall is overmolded with a third elastomeric material.

12. The vial assembly of claim 11, wherein the third elastomeric material is patterned on an exterior surface of the tube wall and comprises at least one textured surface.

13. The vial assembly of claim 11, wherein the third elastomeric material is overmolded around a periphery of the second end and on an exterior surface of the tube wall.

14. The vial assembly of claim 13, wherein the exterior surface of the tube wall comprises at least one strip of the third elastomeric material extending from the second open along the entire length or a portion of the length of the tube wall.

15. The vial assembly of claim 11, wherein the third elastomeric material is identical to or different from the second elastomeric material.

16. The vial assembly of claim 10, wherein the tube wall comprises at least two flanges proximate the flexible bottom, wherein the flanges are configured to hinge inwardly and apply force to the flexible bottom.

17-20. (canceled)

21. A method for removing a frozen sample from a vial assembly comprising a tubular body and a flexible liner inserted into a cavity of the tubular body, the method comprising:
depressing two or more flanges of the flexible liner to apply force to at least a portion of the flexible liner, wherein the flexible liner is configured to compress at least partially upon the application of force.

22. A method for removing a frozen sample from a vial assembly comprising a tubular body comprising a tube wall, a first end, and a flexible bottom disposed proximate the first end, the method comprising:
applying force to at least a portion of the flexible bottom,
wherein the flexible bottom is configured to compress at least partially upon the application of force,
wherein the tube wall and flexible bottom comprise a first material, and
wherein the flexible bottom has a first thickness which is less than a second thickness of the tube wall.

23. The method of claim 22, wherein applying force to at least a portion of the flexible bottom comprises depressing two or more flanges proximate the first end.

24. The method of claim 22, wherein applying force to at least a portion of the flexible bottom comprises manually depressing the flexible bottom.
